(19) United States
(12) Patent Application Publication
(10) Pub. No.: US 2016/0016989 A1
(43) Pub. Date: Jan. 21, 2016

(54) Synthetic Polymers Containing Amino Acid Side Chains

(71) Applicant: CHIMEROCHEM LLC, Portland, OR (US)

(72) Inventor: Theresa Thompson, West Linn, OR (US)

(73) Assignee: Chimerochem LLC, Portland, OR (US)

(21) Appl. No.: 14/768,313

(22) PCT Filed: Mar. 6, 2014
(86) PCT No.: PCT/US14/21076

§ 371 Date: Aug. 17, 2015

Related U.S. Application Data
(60) Provisional application No. 61/786,302, filed on Mar. 14, 2013.

(51) Int. Cl. C07K 1/00; C07F 9/655

(52) U.S. Cl. CPC C07K 1/006; C07F 9/65515

(57) ABSTRACT

A synthetic polymeric molecule containing multiple subunits in which one or more of the subunits contains an amino acid side chain linked to a phosphate or modified phosphate group and is attached to an adjacent subunit by a phosphodiester or modified phosphodiester bond.

This application is a national phase application from PCT Application No. PCT/US2014/021076, which was filed on Mar. 6, 2014 and published as WO 2014/158954, which application claims priority from U.S. Provisional Patent Application No. 61/786,302, filed on Mar. 14, 2013.

### FIELD OF THE INVENTION

The present invention pertains to the field of analogs of polypeptides and to synthetic chemical compounds that are useful in making such analogs.

### BACKGROUND OF THE INVENTION

Polypeptides are polymers of α-amino acids. The general structure of an amino acid is shown below in Formula A.

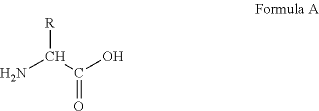

As shown in Formula A, an amino acid contains an amino group (NH₂), a carboxyl group (COOH), and a side chain (R), each of which is attached to a central alpha carbon. There are twenty amino acids (including the imino acid proline) that are encoded by the genetic code, listed below in Table 1 with their respective side chains (R).

TABLE 1

| Amino Acid | Side Chain (R) |
|---|---|
| Alanine | CH3— |
| Arginine | HN═C(NH₂)—NH—(CH₂)₃— |
| Asparagine | H₂N—CO—CH₂— |
| Aspartic Acid | HOOC—CH₂— |
| Cysteine | HS—CH₂— |
| Glutamic Acid | HOOC—(CH₂)₂— |
| Glutamine | H₂N—CO—(CH₂)₂— |
| Glycine | H— |
| Histidine | 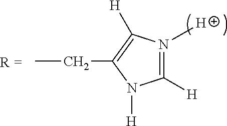 |
| Isoleucine | CH₃—CH₂—CH(CH₃)— |
| Leucine | (CH₃)₂—CH—CH₂— |
| Lysine | H₂N—(CH₂)₄— |
| Methionine | CH₃—S—CH₂—CH₂— |
| Phenylalanine | Phenyl-CH₂— |
| Proline | 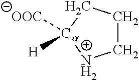 |
| Serine | HO—CH₂— |
| Threonine | CH₃—CH(OH)— |
| Tryptophan | 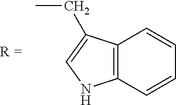 |
| Tyrosine | 4-OH—Phenyl-CH₂— |
| Valine | CH₃—CH(CH₂)— |

In addition to the naturally occurring amino acids, two other amino acids that are not encoded by the genetic code have been found in proteins. Selenocysteine is present in several enzymes, including some that are present in humans. Pyrrolysine is present in some methanogenic archaea in enzymes that they use to produce methane. Additional amino acids that are not DNA encoded include carnitine and GABA (gamma-aminobutyric acid), selenomethionine, and hydroxyproline.

An amino acid may be linked to a second amino acid by a dehydration or condensation reaction in which the hydroxyl group from the carboxy portion of one amino acid and one of the hydrogens from the amino group from the hydroxy portion of a second amino acid are released as water, and a peptide bond (CO—NH) is formed, as shown in Formula B, resulting in the formation of a molecule which is an amide.

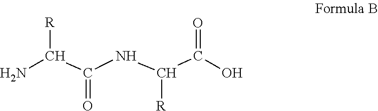

A linkage of two amino acids by a peptide bond is referred to as a dipeptide. A chain of two or more amino acids is referred to as polypeptide. In a polypeptide, each amino acid, except for the two terminal amino acids, is linked to the two adjacent amino acids by polypeptide bonds. In this patent application, the term “polypeptide” refers to a chain of at least two amino acids. If the polypeptide is in a complete biological form and is in a stable conformation, the polypeptide may be referred to herein as a “protein.” The term “peptide,” as used herein, refers to a short amino acid oligomer of 2 to 50 amino acids that may or may not have a stable three-dimensional structure and which may or may not have biological or chemical activity.

Proteins have various functions inside the body of an animal or plant and in the environment. In biological systems, proteins such as collagen, keratin, and plant proteins provide rigidity and form structures. Other proteins are involved in the process of cell signaling and signal transduction. Other proteins, in both biological systems and in the environment, function as enzymes to catalyze chemical reactions.

Cell signaling and signal transduction proteins, such as receptors, receptor ligands, antibodies, and enzymes, have a particular conformation based on precise folding of the polypeptide chain. The amino acids of a polypeptide interact with each other to produce a well-defined three-dimensional structure, the folded protein, known as the native state. It is the sequence of the amino acids in the polypeptide that determines the resulting three-dimensional structure.

In a polypeptide, numerous three-dimensional conformations are possible. However, the conformation that is the most stable thermodynamically is predominately adopted. The peptide bond is rigid and planar. The central carbons of the two amino acids adjacent to a peptide bond, as well as the CO and the NH of the peptide bond itself, lie in a single plane. However, the amino acid side chains (R) are free to rotate around their central carbons. The result is a polypeptide backbone made of a series of rigid planes, with adjacent planes sharing a common point of rotation at the central tetrahedral carbon shared between the adjacent planes. The torsional degrees of freedom at the central carbons the polypeptide chain permits the various amino acid side chains of the polypeptide chain to freely interact with each other and to adopt the most thermodynamically stable conformation, unless prohibited by steric hindrance between atoms.

In polypeptide chains of relatively short length, less than 50 amino acids, and especially those less than 30 amino acids, polypeptides typically have little secondary structure with little folding. However, in polypeptide chains longer than 50 amino acids, secondary structure is of greater significance as the polypeptide adopts a folded conformation that is dependent primarily on the sequence of amino acids, and particularly on their side chains.

The idea to use polypeptides, such as for medical or environmental applications, for their cell signaling, signal transducing, or enzymatic properties, and to a lesser extent for their structural properties, has long been considered to be desirable. However, many problems exist that make such uses of polypeptides an elusive quest.

Chemical synthesis of polypeptides involves the stepwise addition of chemically protected amino acids one-by-one to a growing peptide-bond-linked amino acid chain. Until recently, such chemical synthesis was impractical because the yield at each additional amino acid addition step was too low. Because errors in synthesis of a stepwise addition process are cumulative, for a synthesis method providing a yield of 90% for each additional amino acid, the final yield for a polypeptide of only 10 amino acids would be only 34%, and the final yield for a polypeptide of 20 amino acids would be only 12%. Recently, however, synthetic processes have been reported that provide a yield for each newly added amino acid of greater than 99%, providing for significantly higher yields than were previously obtainable.

A more intractable problem with the use of polypeptides for therapeutic purposes concerns delivery of a polypeptide to its active site and the instability of polypeptides in biologic settings. Because polypeptides are rapidly degraded by salivary and gastric enzymes, oral administration of polypeptides is impractical. However, even when such administration routes are bypassed, such as by intravenous administration, polypeptides are rapidly degraded into inactive fragments by peptidase enzymes located in the bloodstream and throughout the body. Therefore, in the instances in which a polypeptide has been used therapeutically, the rapid degradation and elimination of the polypeptide requires administration of super-pharmacological doses in order to ensure the availability of some active polypeptide, if even for a very brief period of time. Of further concern is that the administration of such super-pharmacological doses of a polypeptide, and of their consequent breakdown products, is likely to be associated with subsequent clinical toxicity. It is primarily for these reasons that the use of polypeptides as therapeutic agents has not been widely utilized.

Similar issues of degradation and delivery occur with the use of polypeptides for environmental purposes. Environmental peptidases, such as those in microscopic organisms such as bacteria and fungi, rapidly degrade polypeptides and render them ineffective for their intended purpose.

Numerous attempts have been made to overcome the problem of instability of environmental and clinically administered polypeptides. As mentioned above, one method is to administer or apply the polypeptide in super-pharmacological doses. Such super-pharmacological dosing, however, is associated with extremely high costs and with possible toxicity. Therefore, in most circumstances, this option is not viable.

Various analogues of polypeptides have been produced in an attempt to obtain a functional protein-like molecule that is resistant to bodily and environmental peptidases. Hechter et al, PNAS, 72(2):563-566 (1975) tested retro-D analogues, peptide analogues in which the CO and the N of the peptide bonds are reversed. As disclosed in Hecther, such peptide analogues would be expected to retain activity when administered orally. However, as further disclosed in Hechter, the retro-D analogues lack functional activity.

Gopi et al, FEBS Letters, 535:175-178 (2003), discloses that the incorporation of a beta-amino acid in a peptide chain provides resistance of the peptide to proteolytic degradation. Natural amino acids are alpha-amino acids in which the amino group and the carboxyl group are attached to the same central carbon atom. In beta-amino acids, the amino and carboxyl groups are attached to different adjacent carbons. Each of the naturally occurring amino acids except glycine is capable of being made as a beta-amino acid. Glycine cannot be made into a beta-amino acid because it has only a single carbon and, therefore, the amino and carboxyl groups cannot be bound to different carbons of a glycine side chain. Gopi disclosed that the addition of a single beta-amino acid into a peptide protects the peptide from degradation by proteases. However, Gopi further disclosed that the presence of the beta-amino acid also considerably reduced the functionality of the peptide.

Hirschmann, U.S. Pat. No. 5,770,732, discloses the replacement of one or more amino acid subunits of an active polypeptide with a pyrrolinone subunit analog of the amino acid. In essence, the amide backbone of a polypeptide is rearranged to replace the central amide group with a 5-membered pyrrolinone ring system. Inventors of the Hirschmann patent formed a company, Provid Pharmaceuticals, Inc. (Piscataway, N.J.), to develop and commercialize peptide analogues containing these pyrrolinone mimetic scaffolds.

To the knowledge of the applicant, none of the peptide analogues of the prior art has found wide-ranging application as surrogates for peptides. It therefore remains an elusive goal to develop a peptide analog that is resistant to protease degradation and that retains the functionality of the native peptide on which the analog Is based.

Nucleic acids are a class of biologic polymers that differ from polypeptides. Unlike polypeptides, monomeric units of nucleic acids are joined together by a backbone of phosphodiester bonds rather than of peptide bonds. Also, unlike polypeptides, naturally occurring nucleic acids contain repeating units of sugars, either ribose in the case of ribonucleic acids (RNAs) or deoxyribose in the case of deoxyribonucleic acids (DNAs).

Another difference between nucleic acids and polypeptides is in the side chains. Whereas polypeptides contain amino acid side chains linked to a central carbon, naturally occurring nucleic acids contain four side chains. In the case of RNA, the side chains are the purines adenine and guanine, and the pyrimidines cytosine and uracil. In the case of DNA, the side chains are the purines adenine and guanine, and the pyrimidines cytosine and thymine.

Aside from these differences, nucleic acids and polypeptides share several features in common. Like polypeptides, single stranded nucleic acids have essentially a planar backbone with high degrees of freedom of motion of the side chains, in this case the purines or pyrimidines. Like polypeptides, nucleic acids are subject to degradation by enzymes within the body and in the environment.

Peptide nucleic acids (PNAs) are chimeric polymeric compounds having a backbone of repeating N-(2-aminoethyl)-glycine units linked by peptide bonds. The various purine and pyrimidine bases are linked to the backbone by methylene carbonyl bonds. PNAs are not degraded by proteases or nucleosidases and are useful to bind to DNA in order to inhibit the action of DNA.

A PNA molecule is a synthetic nucleic acid analogue. To date, the inventor is not aware of a synthetic polypeptide molecule that is based on a phosphodiester or modified phosphodiester backbone. As described in more detail below, such a molecule would be useful for therapeutic and diagnostic indications in human and animal medicine and would be useful in environmental applications in situations where a polypeptide would be useful.

### BRIEF DESCRIPTION OF THE DRAWINGS

FIG. 1 shows the synthesis of an alanine phosphoramidite monomer containing a sugar moiety.

FIG. 2 shows the synthesis of a phenylalanine phosphoramidite monomer containing a sugar moiety.

FIG. 3 shows the synthesis of a cysteine phosphoramidite monomer containing a sugar moiety.

FIG. 4 shows the synthesis of a lysine phosphoramidite monomer containing a sugar moiety.

FIG. 5 shows the synthesis of a tyrosine phosphoramidite monomer containing a sugar moiety.

FIG. 6 shows the synthesis of an alanine phosphoramidite monomer lacking a sugar moiety.

FIG. 7 shows the synthesis of a phenylalanine phosphoramidite monomer lacking a sugar moiety.

FIG. 8 shows the synthesis of a lysine phosphoramidite monomer lacking a sugar moiety.

FIG. 9 shows the synthesis of a tyrosine phosphoramidite monomer lacking a sugar moiety.

### DETAILED DESCRIPTION OF THE INVENTION

In one embodiment, the invention is a synthetic polymeric molecule containing elements of both polypeptides and nucleic acids. The polymeric molecule contains a series of monomer subunits that are linked in a chain by a phosphodiester, or modified phosphodiester, backbone such as is present in nucleic acids or backbone-modified nucleic acids. The synthetic polymeric molecule has increased acid, nuclease, and/or protease stability as compared to a native polypeptide or oligonucleotide. The monomers of the molecule also contain an amino acid side chain in place of the bases present in nucleic acids.

The monomers of this application allow for the synthesis of long polymers with high yields because the synthesis of such polymers may be accomplished by standard DNA oligonucleotide synthetic methods. Thus, an advantage of the present application is improved yield per unit length as compared with automated polypeptide synthesis methods.

In this specification, the term “monomer subunit” refers to a monomer that is present within a chain of subunits in a polymeric molecule, the term “reactive monomer” refers to a molecule that is not part of a polymeric molecule and which may be combined with one or more other reactive monomers to form a polymeric molecule, and the term “monomer” as used herein refers to either or both a monomer subunit and/or a reactive monomer.

In a preferred embodiment, the synthetic polymeric molecule further contains a sugar moiety, such as a pentose sugar like a ribose or deoxyribose. The sugar moiety is connected to, and indirectly links, the amino acid side chain and the phosphate, or modified phosphate, of the phosphodiester, or modified phosphodiester backbone.

In one preferred embodiment, the sugar is deoxyribose and the backbone is phosphodiester. Thus, the polymeric molecule in this embodiment may be considered to be a deoxyribonucleic acid (DNA) analog in which the nitrogenous bases of the nucleosides of the DNA are replaced by amino acid side chains. The polymeric molecule of this embodiment may also be considered to be a polypeptide analog in which the polypeptide backbone has been replaced by a phosphodiester backbone. The DNA/polypeptide analog of this embodiment having a chain of at least 3 analog monomer subunits is shown in Formula C.

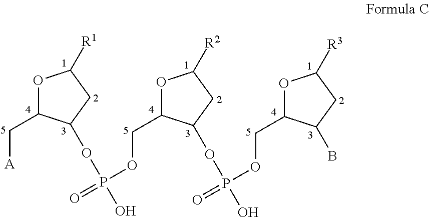

As shown in Formula C, the five carbons of the deoxyribose sugar moiety are numbered 1 to 5. R1, R2, and R3 of Formula C are independently amino acid side chains, which may be of amino acids encoded by the genetic code or of amino acids that are not encoded by the genetic code. Adjacent sugar moieties are linked by phosphodiester bonds as in naturally occurring DNA. A and B of Formula C are not material to this embodiment of the invention and may be, for example, an adjacent monomer subunit, which may or may not be a monomer of this invention. Other examples of suitable A and B groups include H, OH, alkyl groups such as methyl, ethyl, butyl, propyl, or isopropyl groups, alkoxy groups such as methoxy, ethoxy, butoxy, propoxy, or isopropoxy groups, amino groups, carboxy groups, biotin, dyes, a reversed linkage, an amino acid, a polypeptide or analog, a nucleotide, an oligonucleotide, a solid support such as a universal support, and linkages to a solid support such as long chain succinimidyl ester linkage.

Preferably, the amino acid side chain R of Formula C is connected to the sugar at position 1. Less preferably, R is connected to a position on the ring of the sugar other than at position 1, such as at position 2 of a pentose sugar, or at position 2 or 3 of a hexose or heptose sugar.

Reactive groups of the polymeric molecule of Formula C may be in a protected or unprotected state. For example, the potentially reactive O group and OH group of the phosphate of the phosphodiester bond and any reactive groups on the amino acid side chains may be protected. For example, the side chains of alanine, glycine, valine, leucine, and isoleucine are composed of alkyl groups and generally do not require protecting groups to prevent side reactions during chemical synthesis. Similarly, the side chain of phenylalanine contains no reactive functional groups and generally does not require a protecting group. However, the side chains of arginine, asparagine, aspartic acid, cysteine, glutamic acid, glutamine, histidine, lysine, proline, serine, threonine, tryptophan, and tyrosine contain reactive functional groups, and protecting groups are required in order to prevent reactions of these functional groups during chemical synthesis.

Examples of protecting groups that may be utilized include alcohol protecting groups such as acetyl, benzoyl, benzyl, beta-methoxyethoxymethyl ether, dimethoxytrityl (DMT), methoxymethyl ether (MOM), methoxytrityl (MMT), p-methoxybenzyl ether (PMB), methylthimethyl ether, pivaloyl, tetrahydropyranyl (THP), trityl (Tr), silyl ethers such as TMS, TBDMS, TOM, and TIPS, methyl ethers, and ethoxyethyl ethers, amine protecting groups such as carobenzyloxy (Cbz), p-methoxybenzyl carbonyl (Moz or MeOZ), tert-butyloxycarbonyl (BOC), 9-fluroenylmethyloxycarbonyl (FOMC), acetyl (Ac), benzoyl, benzyl, carbamate, p-methoxybenzyl, 3,4-dimethoxybenzyl, p-methoxyphenyl, tosyl, and sulfonamide groups such as Nosyl and Nps groups, carbonyl protecting groups such as acetals and ketals, acylals, and dithianes, carboxylic acid protecting groups such as methyl esters, benzyl esters, tert-butyl esters, esters of 2,6-disubstituted phenols, silyl esters, orthoesters, and oxazoline, terminal alkyne protecting groups such as propargyl alcohols and silyl groups, and phosphate protecting groups such as 2-cyanoethyl and methyl.

In another preferred embodiment, the deoxyribose sugar moiety of one or more of the monomer subunits of Formula C is replaced by a ribose sugar moiety, as shown in Formula D. If desired, the free hydroxyl group of the ribose moiety may be protected.

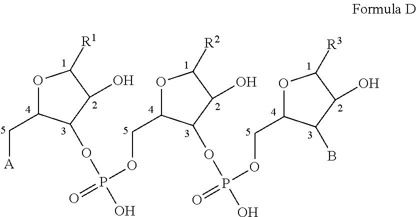

In another embodiment, the sugar moiety of the monomer subunits of the synthetic polymeric molecule is other than deoxyribose or ribose. For example, the sugar moiety may be 2′O-methyl ribose, a triose, a tetrose, a pentose, a hexose, or a heptose moiety, and may be an aldose or a ketose sugar. Examples of suitable sugars include tetroses such as erythrose, threose, and erythrulose; pentoses such as arabinose, lyxose, xylose, ribulose, and xylulose; hexoses such as allose, altrose, glucose, mannose, gulose, idose, galactose, talose, psicose, fructose, sorbose, and tagatose; and heptoses such as sedoheptulose, mannoheptulose, and mannoketoheptose. The sugars may be deoxygenated at one or more positions and thus may be a deoxysugar moiety.

The sugar moieties of the synthetic polymeric molecule may be modified, if desired. For example, any position, such as the 2 position, of the sugar may be halogenated, such as with a fluorine or chlorine. Other modifications include an O-methoxy or ethoxymethoxy in the sugar, such as at the 2 position. Another modification may be a deoxy, such as at position 2 as indicated in Formula C.

In another embodiment, the sugar moiety of the synthetic polymeric molecule is replaced by a ringed structure other than a sugar. For example, the synthetic polymeric molecule may contain a non-sugar, such as a cycloalkyl ring moiety such as cyclopentane or cyclohexane. These ringed structures may include morpholino, piperidino, pyrrolidino, or other ring structures such as those known in instrument based oligonucleotide synthesis. The ringed structure moiety may be modified or substituted as described above for sugars.

In another embodiment, one or more of the monomer subunits of the synthetic polymeric molecule does not include a sugar moiety between the amino acid side chain and the phosphodiester, or modified phosphodiester, group. Instead the sugar moiety is replaced by a linker, as shown below in Formula E.

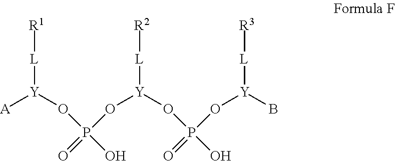

In Formula F, the A, B, and R groups are as in Formulas C, D, and E. In Formula F, however, the sugar moiety is not present. Instead, the synthetic polymeric molecule contains a linker (L) that connects the R group to the phosphodiester backbone of the polymeric molecule and a spacer (Y) that covalently connects adjacent phosphate groups of the phosphodiester backbone of the polymeric molecule.

The linker L is covalently bound to R. The linker L is from 1 to 10 atoms in length and may be constituted of any atom that occurs in biological systems and can form multiple covalent bonds. Thus, examples of the atoms of L include C, N, O, or S. Less preferably, the linker may contain atoms such as Ca, Mn, Mg, Fe, and Se. It is noted that side chains emanating from L are immaterial and are not considered when determining the length of L. If side chains are present on L, the size of side chains is such that the amino acid side chains R of the molecule are accessible for interaction with other compounds, such as for binding.

In a preferred embodiment, L is a covalent chemical linkage resulting from a chemical cross linking reaction that links the activated amino acid side chain R with a Y spacer using a cross linking agent. Examples of such cross linking reagents include homobifunctional and heterobifunctional cross linking reagents such as NHS esters, maleimides, carbodiimides, isothionates, imidoesters, pyridyldithiol, halocetyls, aryl azides, and hydrazides. Other suitable cross linking agents are disclosed in Thermo Scientific Pierce Protein Biologics Products, Product Catalog which may be accessed at www.piercenet.com.

The L as described above may occur as a result of the method of synthesis of the monomers which is disclosed below in the examples in which an amino acid side chain is cross linked to a Y group. There are, however, many methods by which the monomer of this application may be made, some of which do not include the use of a cross linking agent to link R and Y. The monomer resulting from such methods may not have an L, or the L may be other than a covalent chemical linkage resulting from a chemical cross linking reaction.

For example, in the polymeric synthetic molecules shown above in Formulas C and D, the R group is connected to the backbone by a sugar. The portion of the sugar including the O in the ring and the carbons at positions 1 and 2 may be conceived to correspond to L and the carbons at positions 3, 4, and 5 may be conceived to correspond to Y. In this situation, L is not a covalent chemical linkage resulting from a chemical cross linking reaction. Thus, the actual identity of L is not material to the monomer of this application as L is a link between R and the backbone group of the monomer. If desired, the linker L may be omitted, and the amino acid side chain R may be directly connected to Y.

The spacer Y is generally 1 to 15, and preferably 3 to 12, atoms in length. The atoms of Y are sequentially covalently bonded and preferably are C, N, O, or S. Other atoms bonded to the atoms of Y are generally not material, so long as they do not cause steric hindrance when utilizing the molecule. As with L, if side chains are present, or if Y is a portion of a ring structure, such as the ring structure of Formulas C, D, and E, the length of Y is considered to be the number of atoms sequentially between adjacent phosphate groups of the phosphodiester backbone In one preferred embodiment, the synthetic polymeric molecule includes a ring structure of which a portion is a linker L and a portion is a spacer Y. Synthetic polymeric molecules of this application with such a ring structure are shown in Formulas C, D, and E in which the ring structure is a sugar. Alternatively, the ring structure may be other than a sugar moiety. Examples of suitable ring structure moieties that are other than sugars that may be included in the synthetic polymeric molecule and which form both the linker L and the spacer Y are morpholino, cycloalkyl such as cyclobutyl, cyclopentyl, or cyclohexyl, aryl such as phenyl or naphthyl, and heteroaryl such as a sulfur-containing ring like thiophene, a nitrogen-containing ring like pyrrole, imidazole, pyrazole, pyrroline, pyrrolidine, pyridine, pyrimidine, purine, quinoline, isoquinoline, or carbazole, or an oxygen-containing ring like furan, or a combination such as oxazole or thiazole. The ring structure, whether a sugar or non-sugar moiety, may be substituted. Thus, the ring structure may include groups such as alkyl groups, amino groups, mercapto groups, or halogen groups, such as chlorine or fluorine.

To this point in this specification, all of the above embodiments of the polymeric molecule are disclosed with a phosphodiester backbone. In nucleic acid applications, various changes in the phosphodiester backbone have been introduced for various reasons, such as to facilitate synthesis or to render the backbone more resistant to degradation. Such changes in the phosphodiester backbone may be utilized in the polymeric molecule of this application.

Any modifications of the phosphodiester backbone that are known in the field of nucleic acid backbone chemistry may be utilized for the monomer of the present application. For example, in place of a phosphodiester linkage, the backbone may contain a phosphorothioate or a phosphorodithioate linkage in which either or both of the non-bridging oxygens (O) is replaced by a sulfur (S). The backbone may contain a phosphorothiolate or diphosphorothiolate linkage in which either or both of the bridging O groups is replaced with an S. The backbone may include alkylphosphonate, such as a methylphosphonate or ethylphosphonate in which either or both non-bridging O groups is replaced with an alkyl, such as a methyl or ethyl, group. The backbone may contain an alkoxyphosphonate linkage, such as a methoxyphosphonate or an ethoxyphosphonate, in which either or both non-bridging O groups is replaced with an alkoxy, such as methoxy or ethoxy, group. The backbone may contain a phosphoramidate linkage in which one or more of the bridging and/or non-bridging O groups is replaced with an amino group. The above are only examples of modifications of the phosphodiester backbone that may be utilized.

Thus, as described above, the synthetic polymeric molecule has the generic formula shown below as Formula G.

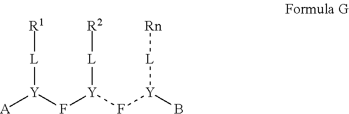

In Formula G, the variables A, B, R, L, and Y are as in Formulas C to F. F is a phosphodiester or modified phosphodiester backbone group, and n=at least 2. The dashed lines in Formula G indicate that the polymeric molecule of Formula G contains at least two subunits and that further subunits are optional.

The synthetic polymeric molecule contains multiple subunits, at least one of which is represented in Formula H wherein, A, B, Y, L, F, and R are as above in Formulas C to G.

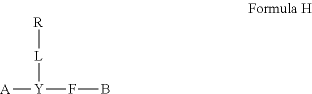

In a preferred embodiment, the polymeric molecule of this application contains subunits, as shown in Formula H, and each subunit of the synthetic polymeric molecule is as shown in Formula H. However, and as discussed in more detail below, the polymeric molecule of this application may contain subunits other than those shown in Formula H.

For example, the polymeric molecule may contain one or more subunits as shown in Formula H and one or more subunits that are nucleotides, such as of DNA or RNA. The backbone group of the nucleotide may be a phosphodiester backbone group or a modified phosphodiester backbone group. Thus, in one embodiment, the polymeric molecule contains one or more subunits that are nucleosides connected to a phosphodiester, or modified phosphodiester backbone group.

As with the monomer subunits shown in Formula H, the subunits that are other than those of Formula H may be protected by the presence of protecting groups. Such protecting groups are known in the art.

In a preferred embodiment, the monomer of this application contains an amino acid side chain R, a linker L, a spacer Y, and a phosphodiester or modified phosphodiester group. In one preferred embodiment, the monomer of this application is as is shown in Formula I.

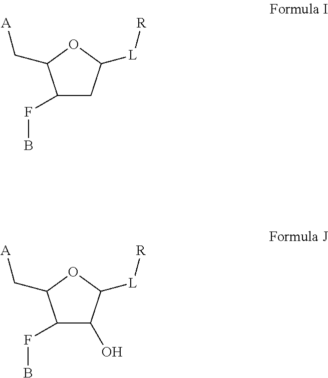

In Formula I, the spacer Y and a portion of the linker L are within a deoxyribose sugar, R is an amino acid side chain and A and B independently may be an adjacent monomer subunit, which may or may not be a monomer of this invention. Other examples of suitable A and B groups include H, OH, alkyl groups such as methyl, ethyl, butyl, propyl, or isopropyl groups, alkoxy groups such as methoxy, ethoxy, butoxy, propoxy, or isopropoxy groups, amino groups, carboxy groups, biotin, dyes, a reversed linkage, an amino acid, a polypeptide or analog, a nucleotide, an oligonucleotide, a solid support such as a universal support, and linkages to a solid support such as long chain succinimidyl ester linkage.

In another preferred embodiment, the monomer contains a ribose moiety, as shown in Formula J in place of the deoxyribose moiety shown in Formula I.

In another preferred embodiment, the ribose or deoxyribose moiety of Formula I or J is replaced with a sugar moiety other than deoxyribose or ribose, as shown in Formula K. For example, the sugar moiety may be 2′O-methyl ribose, a triose, a tetrose, a pentose, a hexose, or a heptose moiety, and may be an aldose or a ketose sugar. Examples of suitable sugars include tetroses such as erythrose, threose, and erythrulose; pentoses such as arabinose, lyxose, xylose, ribulose, and xylulose; hexoses such as allose, altrose, glucose, mannose, gulose, idose, galactose, talose, psicose, fructose, sorbose, and tagatose; and heptoses such as sedoheptulose, mannoheptulose, and mannoketoheptose. The sugars may be deoxygenated at one or more positions and thus may be a deoxysugar moiety.

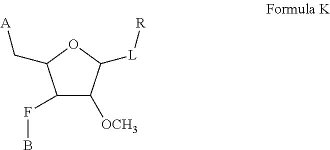

The sugar moieties of the monomer, whether as a monomer subunit or as a reactive monomer, may be modified, if desired. For example, any position, such as the 2 position, of the sugar may be halogenated, such as with a fluorine or chlorine. Other modifications include an O-methoxy or ethoxymethoxy in the sugar, such as at the 2 position. Another modification may be a deoxy, such as at position 2 as indicated in Formula I.

In another embodiment, the sugar moiety of the monomer is replaced by a ringed structure other than a sugar, as shown in Formula L. For example, the monomer may contain a non-sugar, such as a cycloalkyl ring moiety such as cyclopentane or cyclohexane. The ringed structure moiety may be modified or substituted as described above for sugars.

In another embodiment, the sugar moiety or ringed structure is not present in the monomer and, in its place, the monomer contains a linker L and a spacer Y, wherein L connects the amino acid side chain R to the spacer and the spacer is a series of atoms that connects A and F and attaches to L. This embodiment is shown in Formula L.

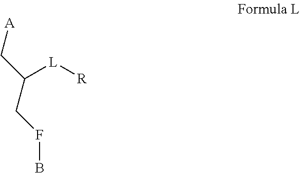

In Formula L, the linker L is covalently bound to R. The linker L is from 1 to 10 atoms in length and may be constituted of any atom that occurs in biological systems and can form multiple covalent bonds. Thus, examples of the atoms of L include C, N, O, or S. Less preferably, the linker may contain atoms such as Ca, Mn, Mg, Fe, and Se. It is noted that side chains emanating from L are immaterial and are not considered when determining the length of L. If side chains are present on L, the size of size chains is such that the amino acid side R of the molecule are accessible for interaction with other compounds, such as for binding.

In a preferred embodiment, L is a covalent chemical linkage resulting from a chemical cross linking reaction that links the activated amino acid side chain R with a Y spacer using a cross linking agent. In Formula L the Y spacer is a non-sugar spacer. As shown in L, Y is a three carbon spacer. Accordingly, the Y letter is not explicitly shown above in Formula L. Examples of such cross linking reagents include homobifunctional and heterobifunctional cross linking reagents such as NHS esters, maleimides, carbodiimides, isothionates, imidoesters, pyridyldithiol, halocetyls, aryl azides, and hydrazides. Other suitable cross linking agents are disclosed in Thermo Scientific Pierce Protein Biologics Products, Product Catalog which may be accessed at www.piercenet.com.

The L as described above generally occurs as a result of the method of synthesis of the monomer which is disclosed below in the examples in which an amino acid side chain is cross linked to a Y group. There are, however, many methods by which the monomer of this application may be made, some of which do not include the use of a cross linking agent to link R and Y. The monomer resulting from such methods may not have an L, or the L may be other than a covalent chemical linkage resulting from a chemical cross linking reaction.

For example, in the polymeric synthetic molecules shown above in Formulas C and D, the R group is connected to the backbone by a sugar. The portion of the sugar including the O in the ring and the carbons at positions 1 and 2 may be conceived to correspond to L and the carbons at positions 3, 4, and 5 may be conceived to correspond to Y. In this situation, L is not a covalent chemical linkage resulting from a chemical cross linking reaction. Thus, the actual identity of L is not material to the monomer of this application as L is a link between R and the backbone group of the monomer. If desired, the linker L may be omitted and the amino acid side chain R and Y may be directly connected to each other.

In the above embodiments of the monomer, the monomer contains a phosphodiester group. In nucleic acid applications, various changes in the phosphodiester backbone have been introduced for various reasons, such as to facilitate synthesis or to render the backbone more resistant to degradation. Such changes in the phosphodiester backbone may be utilized in the polymeric molecule of this application and modified phosphodiester groups may be utilized in the monomer.

Any modifications of the phosphodiester backbone that are known in the field of nucleic acid backbone chemistry may be utilized for the monomer of the present application. For example, for a reactive monomer, F in any of Formulas H through L may be a phosphoramidate, a phosphoramidite such as p-ethoxy, or a phosphonate group. For example, for a monomeric subunit, F may be a phosphodiester group, a phosphorothioate or a phosphorodithioate group, a phosphorothiolate or diphosphorothiolate group, an alkylphosphonate, such as a methylphosphonate or ethylphosphonate group, an alkoxyphosphonate, such as a methoxyphosphonate or an ethoxyphosphonate group, an alkoxy, such as methoxy or ethoxy group, a phosphoramidate group, or other modifications of phosphodiester groups as used in nucleic acid chemistry.

In the above embodiments of the monomer, any amino acid side chain may be included. However, because the side chain of the amino acid glycine is simply a hydrogen (H), the reactive monomer containing a glycine side chain as R is excluded from the scope of this application. The monomer subunit containing a glycine side chain, however, is not excluded from the scope of this application.

Reactive groups of the monomer, whether as a reactive monomer or as a monomer subunit, may be in a protected or unprotected state. Such reactive groups may include, for example, imine groups, amine groups, hydroxyl groups, thiol, and carboxyl groups. The side chains of alanine, glycine, valine, leucine, and isoleucine are composed of alkyl groups and generally do not require protecting groups to prevent side reactions during chemical synthesis. Similarly, the side chain of phenylalanine contains no reactive functional groups and generally does not require a protecting group. However, the side chains of arginine, asparagine, aspartic acid, cysteine, glutamic acid, glutamine, histidine, lysine, proline, serine, threonine, tryptophan, and tyrosine contain reactive functional groups, and protecting groups are required in order to prevent reactions, such as branching, of these functional groups during chemical synthesis.

Examples of protecting groups that may be utilized in the monomer of this application include alcohol protecting groups such as acetyl, benzoyl, benzyl, beta-methoxyethoxymethyl ether, dimethoxytrityl (DMT), methoxymethyl ether (MOM), methoxytrityl (MMT), p-methoxybenzyl ether (PMB), methylthimethyl ether, pivaloyl, tetrahydropyranyl (THP), trityl (Tr), silyl ethers such as TMS, TBDMS, TOM, and TIPS, methyl ethers, and ethoxyethyl ethers, amine protecting groups such as carobenzyloxy (Cbz), p-methoxybenzyl carbonyl (Moz or MeOZ), tert-butyloxycarbonyl (BOC), 9-fluroenylmethyloxycarbonyl (FOMC), acetyl (Ac), benzoyl, benzyl, carbamate, p-methoxybenzyl, 3,4-dimethoxybenzyl, p-methoxyphenyl, tosyl, and sulfonamide groups such as Nosyl and Nps groups, carbonyl protecting groups such as acetals and ketals, acylals, and dithianes, carboxylic acid protecting groups such as methyl esters, benzyl esters, tert-butyl esters, esters of 2,6-disubstituted phenols, silyl esters, orthoesters, and oxazoline, terminal alkyne protecting groups such as propargyl alcohols and silyl groups, and phosphate protecting groups such as 2-cyanoethyl and methyl.

The synthetic polymeric molecule of this application is preferably made by solid state phosphoramidite synthesis methods that are used to synthesize nucleic acids such as DNA and RNA. In such methods, a first monomer is anchored to a solid state support such as a controlled pore glass bead (CPG). As in phosphoramidite synthesis schemes, the monomer preferably has a protecting group covering each reactive group of the monomer.

In a first step of elongation, the existing terminal monomer is deblocked, which removes the blocking groups such as DMT from the site of chain elongation and leaves a reactive group at that position. An activator solution is added to the new monomer. Next, the new monomer to be added to the chain is combined with the bound deblocked monomer or chain, thereby extending the chain. After allowing the reaction to extend the chain by one monomer, the next step is a capping step whereby unreacted reagents are rendered inactive, thereby preventing elongation of chains with internal deletions. Next an oxidation step occurs whereby an O or an S is added to the phosphate group to yield a phosphodiester, phosphorothioate, or other modified phosphorus linkage. The cycle is repeated for additional monomeric units until the desired polymeric molecule is built. Following the completion of all monomer additions, the molecule is cleaved from the support and deprotected.

A general scheme for making the monomers is analogous to the process described in Caruthers, U.S. Pat. No. 4,415,732 in Example I, with the exception that B in the Caruthers example differs from the present application.

The monomers of the invention may be made as follows. A protected chemical group corresponding to the side chain of amino acid is obtained. In cases where the side chain contains an amino, hydroxy, carboxy, or thiol reactive group, the reactive group is protected with a chemical protecting group that is preferably compatible with DNA phosphoramidite chemistry. Compatibility with DNA phosphoramidite chemistry is preferred because this will permit the monomer to be incorporated into a polymer by such chemistry. However, if the monomer is to be used by itself, without being incorporated into a polymer, or if the monomer is to be incorporated into a polymer by other than DNA phosphoramidite chemistry, a chemical protecting group that is not compatible with DNA phosphoramidite chemistry may be used. The protected amino acid side chain is reacted with a sugar or other L group as discussed above in order to obtain an intermediate that is to be combined with a phosphate, or modified phosphate, group to provide the monomer. A blocking group, such as DMT, is used in order to prevent unwanted side reactions on the sugar or other L group. The blocked intermediate is reacted with a hetero- or homo-bifunctional cross-linking agent such as EDC, NHS ester, or other agent such as those referred to in the Thermo Scientific Pierce Protein Biologics Products, Product Catalog. The blocked intermediate is then combined with a phosphitylating agent, such as chloro-N N-dimethylaminomethoxyphosphine [CH₃O—P(Cl)—N(CH₃)₂], to produce the monomer.

The synthetic polymeric molecule of this application may be used to mimic or to modulate the action of a polypeptide. As used herein, the term “mimic” means to utilize the synthetic polymeric molecule to obtain the response, irrespective of amplitude of activity or time course, that would otherwise be obtained by using a polypeptide. In many instances, the synthetic polymeric molecule will have a sequence of monomers with amino acid side chains that corresponds to the amino acid sequence of a polypeptide. As used herein, the term “modulate” means to inhibit or stimulate or otherwise modify the activity of a polypeptide. For example, the synthetic polymeric molecule may increase or decrease the effect of a polypeptide through direct catalysis or through disruption of enzyme polymerization, folding, binding to cofactors, or binding to substrate.

An example of a polypeptide that may be mimicked by the synthetic polymeric compound of this application is cholecystokinin, a polypeptide that aids in transporting nutrients through the wall of the duodenum. A synthetic polymeric molecule having a series of monomers containing amino acid side chains that correspond sequentially to the amino acid sequence of cholecystokinin may be administered orally. Because the synthetic polymeric molecule is resistant to degradation by proteolytic enzymes in the gastrointestinal tract, it will reach the duodenum after passing through the stomach and will bind to gut wall to increase uptake of nutrients in the duodenum.

Another example of a polypeptide that may be mimicked is ACTH (adrenocorticotropic hormone). Injection of a synthetic polymeric molecule containing a sequence of monomers of the present application with amino acid side chains that correspond to the amino acid sequence of ACTH, or exposure of adrenal cells in culture to the synthetic polymeric molecule, will cause an increase in secretion of cortisol by adrenal cells.

A third example of a polypeptide that may be mimicked in accordance with this application is systemin, a hormone that is secreted by plants to defend against insect predation. Applying to plants a synthetic polymeric molecule having a monomer sequence with amino acid side chains that correspond to the amino acid sequence of systemin increases the resistance of the plants to insect infestation.

A fourth example of a polypeptide that may be mimicked in accordance with this application is phytosulfokine (PSK). PSK is a 5-mer polypeptide that promotes cellular differentiation in asparagus and carrots.

An example of a polypeptide that may be modulated is histamine. Administration of a synthetic polymeric molecule containing a sequence of monomers having amino acid side chains that correspond to the amino acid sequence of histamine results in a decrease due to competitive binding of the histamine receptor.

The synthetic polymeric molecule may be formulated by any means known in the art, including but not limited to tablets, capsules, caplets, suspensions, powders, lyophilized forms and aerosols, and may be mixed and formulated with buffers, binders, stabilizers, anti-oxidants and other agents known in the art. The formulation containing the synthetic polymeric molecule may be administered to an individual by any means known in the art, including but not limited to intravenous injection, subcutaneous injection, administration through mucous membranes, oral administration, dermal administration, skin patches, and aerosols. The formulation containing the synthetic polymeric molecule may be applied to the environment, such as to plants, by any means known in the art, such as by spraying, painting, dabbing, and applying in the form of granules.

In one embodiment, the present invention is a pharmaceutical composition that includes the synthetic polymeric molecule and a pharmaceutically acceptable carrier. The synthetic polymeric molecule may be formulated or compounded into pharmaceutical compositions that include at least one synthetic polymeric molecule of this application together with one or more pharmaceutically acceptable carriers, including excipients, such as diluents, carriers and the like, and additives, such as stabilizing agents, preservatives, solubilizing agents, buffers and the like, as may be desired. Formulation excipients may include polyvinylpyrrolidone, gelatin, hydroxy cellulose, acacia, polyethylene glycol, mannitol, sodium chloride or sodium citrate. For injection or other liquid administration formulations, water containing at least one or more buffering constituents is suitable, and stabilizing agents, preservatives and solubilizing agents may also be employed. For solid administration formulations, any of a variety of thickening, filler, bulking and carrier additives may be employed, such as starches, sugars, fatty acids and the like. For topical administration formulations, any of a variety of creams, ointments, gels, lotions and the like may be employed. For most pharmaceutical formulations, non-active ingredients will constitute the greater part, by weight or volume, of the preparation. For pharmaceutical formulations, it is also contemplated that any of a variety of measured-release, slow-release or time-release formulations and additives may be employed, so that the dosage may be formulated so as to effect delivery of a peptidomimetic compound of this application over a period of time.

The synthetic polymeric molecules and pharmaceutical compositions of this application may be administered by injection, which injection may be intravenous, subcutaneous, intramuscular, intraperitoneal, or by any other means known in the art. In general, any route of administration by which the synthetic polymeric molecules may be introduced into the body may be employed. Administration means may include administration through mucous membranes, buccal administration, oral administration, dermal administration, inhalation administration, nasal administration and the like. The dosage for treatment is administration, by any of the foregoing means or any other means known in the art, of an amount sufficient to bring about the desired therapeutic effect.

The monomers of this application have several uses. Primarily, the monomers are useful as building blocks for making the polymers of the invention which are useful as peptide or protein mimetics or modulators. Additionally, the monomers are useful as tags or reporting groups on nucleic acid molecules.

The monomers of this application provide several advantageous features when used as a tag or reporting group. Because they may be inserted into a synthetic nucleic acid by standard phosphoramidite chemistry such as is used to synthesize nucleic acids, they can be inserted into any position of a nucleic acid. In addition, multiple copies of the monomer, or different monomers having varying amino acid side chains, may be utilized, providing a unique label. Additionally, the monomers may be used in combination with other oligonucleotide labels such as fluorescence or metal labeling.

The invention is further illustrated in the following non-limiting examples.

### Example 1

### Preparation of Monomers

Amino acids are grouped according to complexity of amino acid side chains as shown in Table 2.

TABLE 2

| | |
|---|---|
| Very Complex Amino Acid Side Chains | Arg, Pro, Trp |
| Complex Amino Acid Side Chains | Asn, Cys, Gin, Lys, |
| | Met, Phe, Tyr, His |
| Simple Amino Acid Side Chains | Ala, Asp, Glu, Ile, |
| | Leu, Ser, Thr, Val, Gly |

The simple amino acid side chains lack reactive groups such as —OH, —NH₂, —COOH, —SH, and includes side chains of alanine, aspartic acid, glutamic acid, isoleucine, leucine, serine, threonine, valine, and glycine. Complex amino acid side chains include the side chain of phenylalanine, which lacks reactive groups, and amino acid side chains of asparagine, cysteine, glutamine, lysine, methionine, tyrosine, and histidine. Very complex amino acid side chains contain multiple reactive groups and include those of arginine, proline, and tryptophan.

### Example 1a

### Preparation of Deoxyribophosphoramidite Monomers with Simple Amino Acid Side Chains

### Example 1a.1

### Alanine

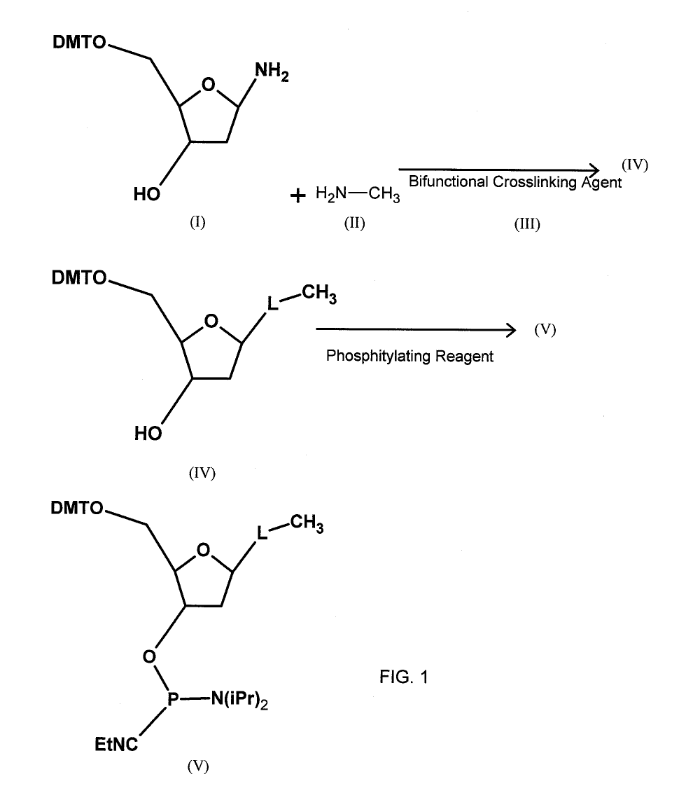

A phosphoramidite monomer is prepared with the side chain of the amino acid alanine covalently bound to the backbone spacer. In this example, as shown in FIG. 1, compound one (I) is the backbone spacer, a 5′ dimethoxytrityl, 1′ amine modified cyclic deoxyribose (I). Compound two (II) is methoxyamine (CAS#74-89-5, compound ID 6329). Compounds I and II are crosslinked using a bifunctional crosslinking agent (III) such as DMA (Dimethyl adipimidate, CAS #14620-72-5)(Thermo Scientific part#20660) as suggested by the manufacturer. The crosslinked intermediate (IV), 1 mmole, is dissolved in 3 ml dry, acid free CHCl₃ and diisopropylethylamine, 4 mmole, in a 10 ml reaction vessel preflushed with dry nitrogen. Phosphitylating reagent (n-dimethylaminomethoxy-phosphine, 2 mmole) is added dropwise to the reaction vessel while held under dry nitrogen. This phosphitylating protocol, as described by Carruthers (U.S. Pat. No. 4,415,732) requires that after a reaction time of at least 15 minutes under dry nitrogen the solution is transferred with 35 ml of ethyl acetate into a 125 ml separatory funnel. The solution is then extracted four times with 80 ml saturated NaCl. The organic phase is then dried over anhydrous Na₂SO₄ and evaporated to a foam under reduced pressure. The foam is then dissolved with either 10 ml toluene or 10 ml ethyl acetate. The solution is then added dropwise to 50 ml of cold (−78 degrees C) hexanes, with vigorous stirring to produce a powder. The suspension is filtered and the powder washed with a further 75 ml of cold hexanes. The resulting powder is then dried under reduced pressure and dry nitrogen with the dried product stored under dry nitrogen.

### Example 1a.2

### Aspartic Acid

A monomer is prepared according to Example 1a.1 except that the starting material II is the side chain of aspartic acid, 3-aminopropanoic acid CID#239.

### Example 1a.3

### Glutamic Acid

A monomer is prepared according to Example 1a.1 except that the starting material II is the side chain of glutamic acid, aminobutanoic acid CID#119.

### Example 1a.4

### Isoleucine

A monomer is prepared according to Example 1a.1 except that the starting material II is the side chain of isoleucine, 2-amino butane CID#2724537.

### Example 1a.5

### Leucine

A monomer is prepared according to Example 1a.1 except that the starting material II is the side chain of leucine, iso-butylamine CID#6558.

### Example 1a.6

### Serine

A monomer is prepared according to Example 1a.1 except that the starting material I is the side chain of serine, ethanolamine CID #700.

### Example 1a.7

### Threonine

A monomer is prepared according to Example 1a.1 except that the starting material II is the side chain of threonine, 3-amino-2-propanol, CID#4631415.

### Example 1a.8

### Valine

A monomer is prepared according to Example 1a.1 except that the starting material II is the side chain of valine, 2-propylamine CID#6363.

### Example 1a.9

### Glycine

For the glycine-based monomer, existing monomers that are utilized for automated oligonucleotide synthesis, such D-spacer (Glen Research catalog #10-1914, Sterling Va.) may be utilized.

### Example 1b

### Preparation of Deoxyribophosphoramidite Monomers with Complex Amino Acid Side Chains

### Example 1b.1

### Phenylalanine

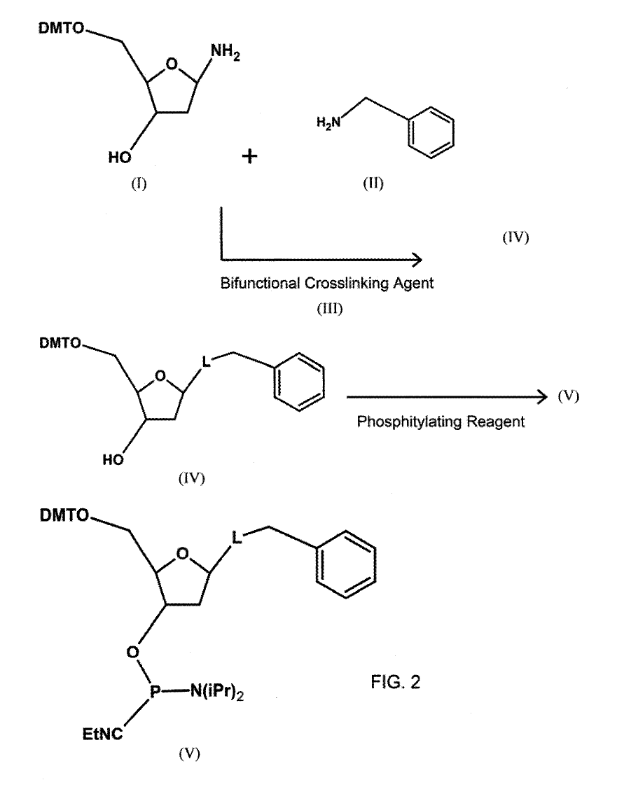

A monomer is prepared according to Example 1a.1 except that the starting material II is the side chain of phenylalanine, benzylamine CID #7504 as shown in FIG. 2.

### Example 1b.2

### Asparagine

A monomer is prepared according to Example 1b.2 except that the starting material II is L-asparagine, CID#6267. The asparagine amino acid is protected with an acetyl group on the primary amine as described for L-arginine (example 1c.1).

### Example 1b.3

### Cysteine

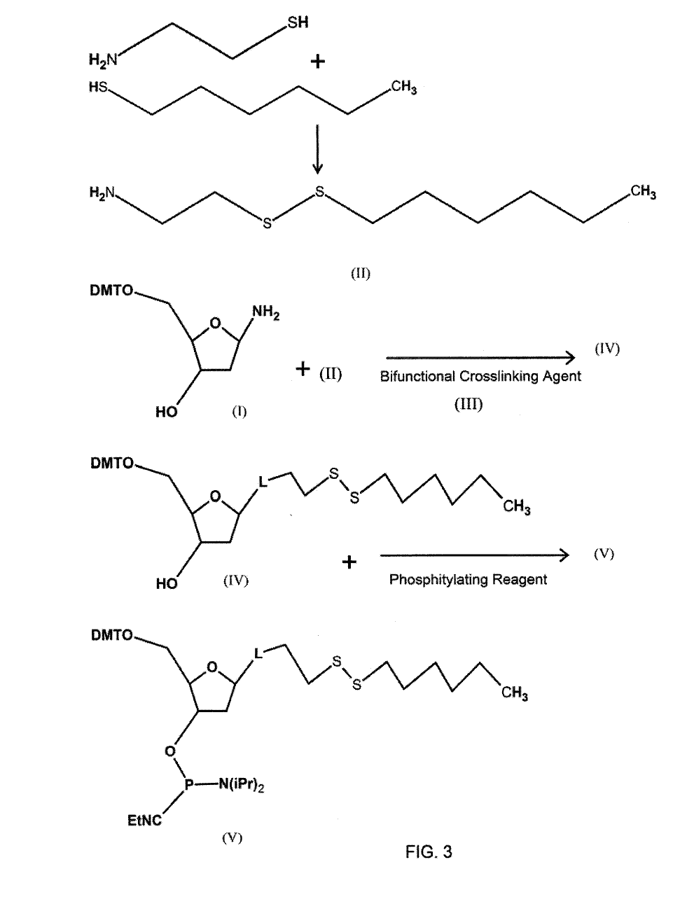

As shown in FIG. 3, A monomer is prepared according to Example 1b.2 except that the starting material is the side chain of cysteine, 2-aminoethanethiol CID#6058. (1-mercaptohexane CID #8106 is used as a protecting group for thiol of cysteine.)

### Example 1b.4

### Glutamine

A monomer is prepared according to Example 1b.2 except that the starting material II is L-glutamine CID#5961. The L-glutamine amino acid is protected with an acetyl group on the primary amine as described for L-arginine (example 1c.1).

### Example 1b.5

### Lysine

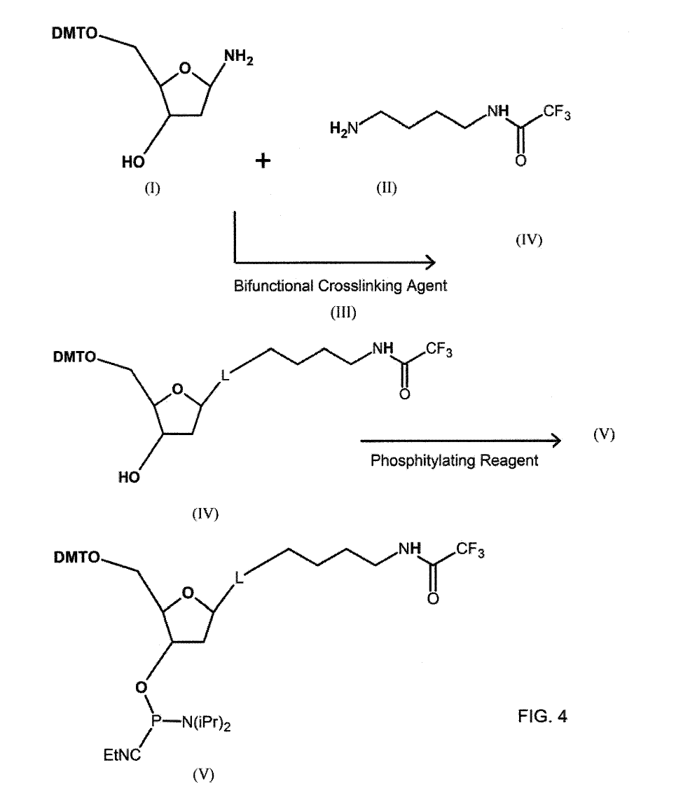

As shown in FIG. 4, a monomer is prepared according to Example 1b.2 except that the starting material II has the side chain of lysine, 4-aminobutanoic acid CID #119. The amino group of 4-aminobutanoic acid is protected with an acetyl group on the primary amine as described for L-arginine (example 1c.1).

### Example 1b.6

### Methionine

A monomer is prepared according to Example 1b.2 except that the starting material II has the side chain of methionine, 3-methylthiopropylamine CID#77743.

### Example 1b.7

### Tyrosine

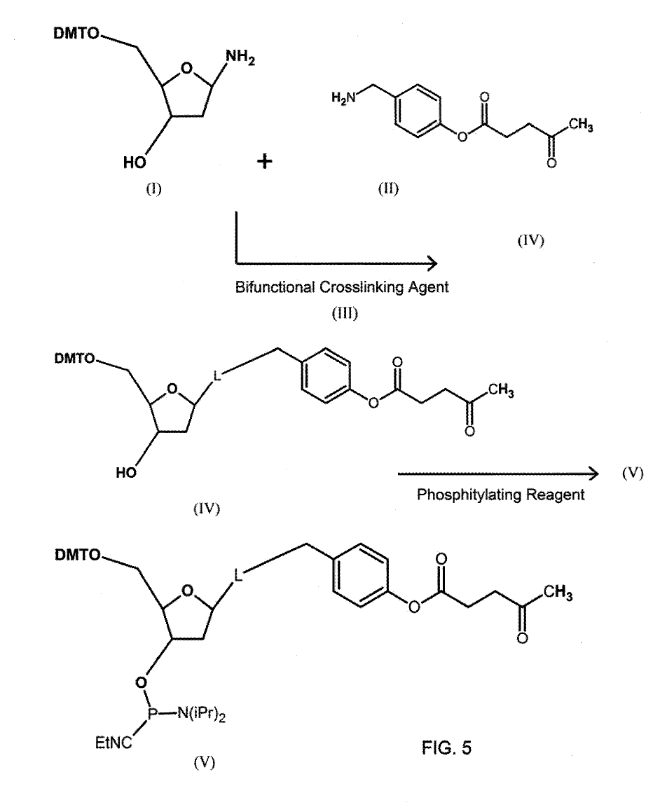

A monomer is prepared according to Example 1b.2 except that the starting material II has the side chain of tyrosine, 4-hydroxybenzylamine CID #97472, as shown in FIG. 5.

### Example 1c

### Preparation of Deoxyribophosphoramidite Monomers with Very Complex Amino Acid Side Chains

### Example 1c.1

### Arginine

L-arginine CID #6322 (10 mg/mml in methanol is reacted with acetyl anhydride at a fourfold molar excess of acetic anhydride to primary amines in the sample. The reaction continues at room temperature in a fume hood for 24 hours with continuous mixing.

### Example 1c.2

### Histidine

A monomer is prepared according to Example 1c.1 except that the starting material II is L-histidine CID #6274. The L-histidine amino acid is protected with an acetyl group on the primary amine as described for L-arginine (example 1c.1).

### Example 1c.3

### Proline

A phosphoramidite monomer was prepared with the side chain of the amino acid proline covalently bound to the backbone spacer. In this example, compound one is the backbone spacer, a 5′ dimethoxytrityl, 1′ amine modified cyclic deoxyribose (I). Compound two (II) is proline (CAS#7005-20-1, compound ID 145742). Compounds I and II are crosslinked using a bifunctional crosslinking agent (III) such as EDC or DCC (Thermo Scientific part numbers 22980 and 20320, respectively) using conditions as suggested by the manufacturer.

### Example 1c.4

### Tryptophan

A monomer is prepared according to Example 1c.1 except that the starting material II is acetyl-L-tryptophan, CID #700653.

### Example 2

### Preparation of Ribophosphoramidite Monomers

The methods of Example 1 are repeated for each amino acid side chain mentioned in Examples 1a.1 to 1c.4 except that ribose sugar moiety is used in place of deoxyribose sugar moiety.

### Example 3

### Preparation of 2′-O-Methyl Ribophosphoramidite

The methods of Example 1 are repeated for each amino acid side chain mentioned in Examples 1a.1 to 1c.4 except that 2′-O-methyl ribose sugar based moiety is used in place of 2′-deoxyribose based sugar. In both cases the 5′hydroxyl group is blocked with a DMT group (dimethoxytrityl).

### Example 4

### Preparation of Phosphoramidate Monomers Lacking Sugar Moiety

The methods of Example 1 are repeated for each amino acid side chain mentioned in Examples 1a.1 to 1c.4 except that the amino acid side chain is connected through an L group to a Y spacer (compound I Example 4a) separating the betacyanoethylphosphoramidite group and the DMT protected oxygen where the spacer is other than a sugar.

### Example 4a

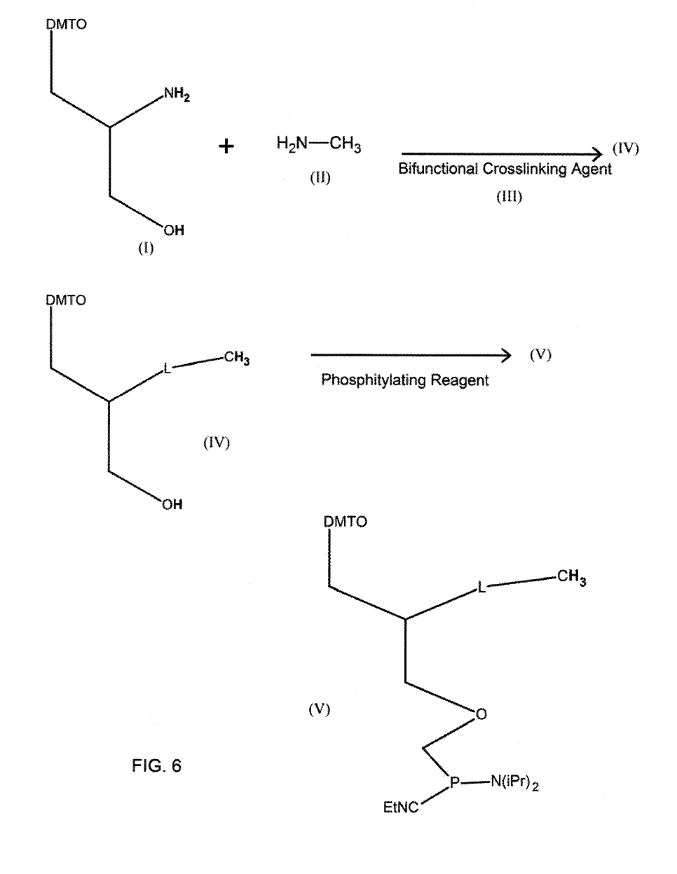

### Alanine, shown in FIG. 6

### Example 4b

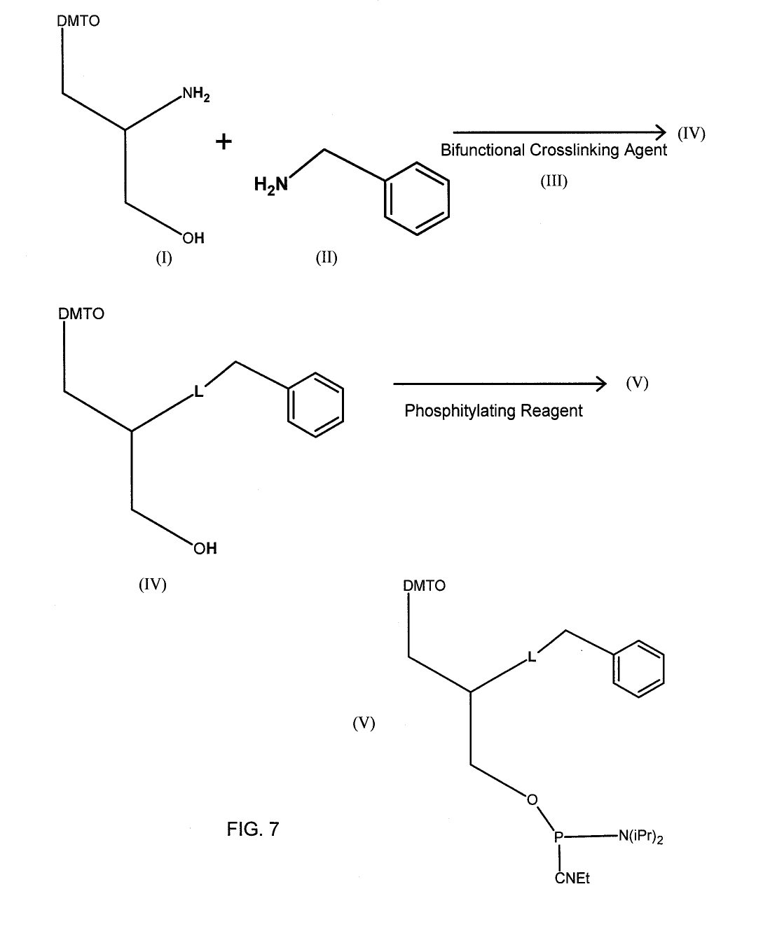

### Phenylalanine, shown in FIG. 7

### Example 4c

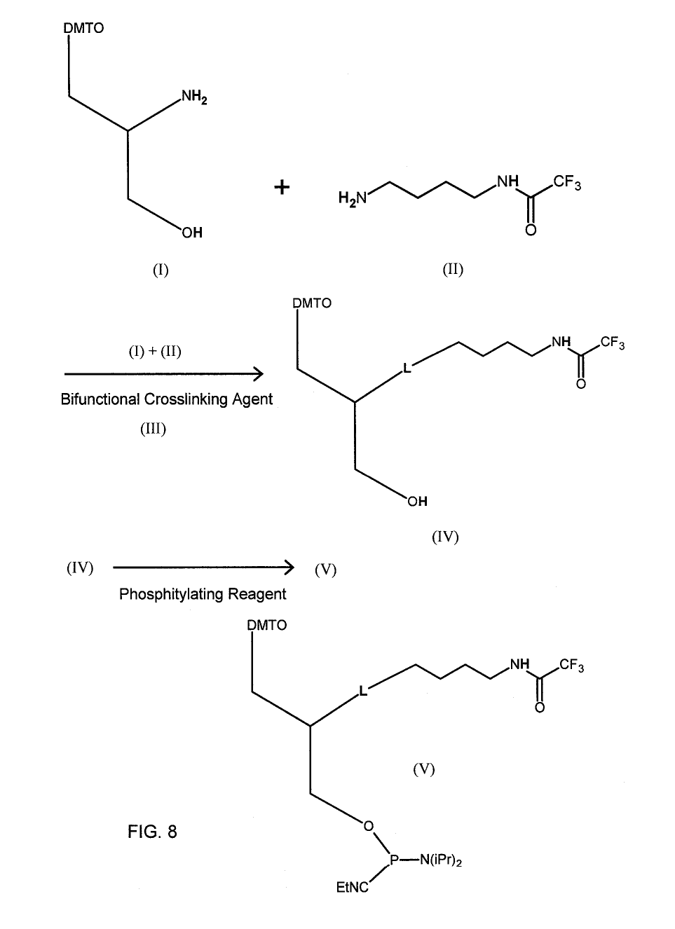

### Lysine, as shown in FIG. 8

### Example 4d

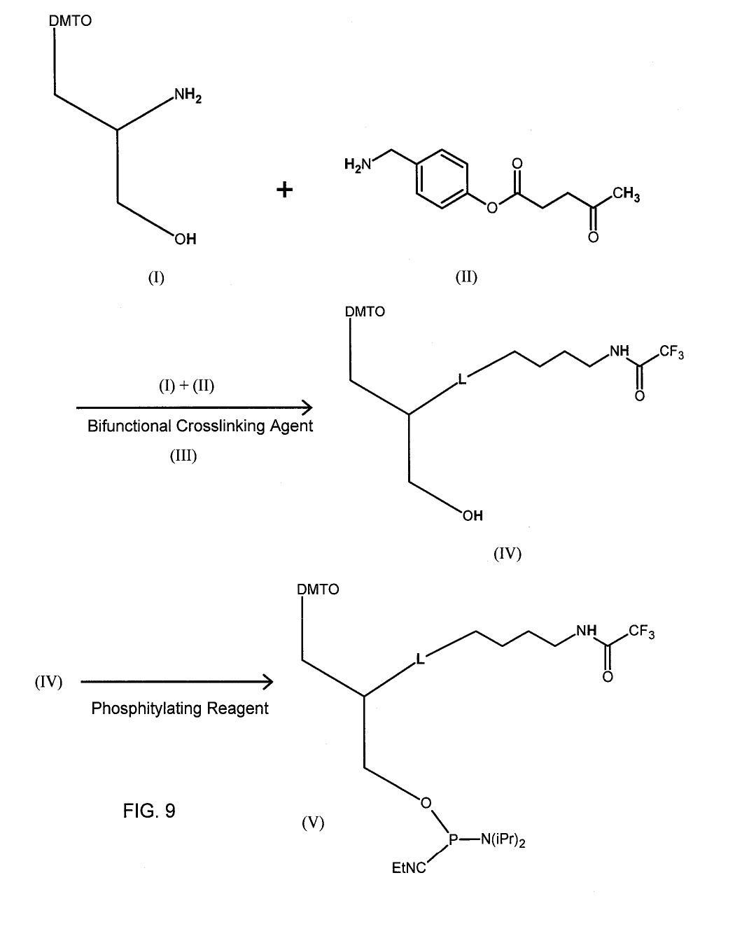

### Tyrosine, as shown in FIG. 9

### Example 5

### Alternate Method for Making Monomers Wherein the Final Monomer Lacks an L Group

The starting material is methylpropanediol CID #7503. The methylpropanediol (10 mmol) is reacted with dimethoxytrityl chloride (12 mmol) in the presence of triethylamine (25 mmol) and reacted as per Zekri et al. (Zekri, Alamdari, and Khalafi-Nezhad. 2010. Bull. Chem. Soc. Ethiop. 24(2): 299-304). The cooled reaction mixture was dissolved in 100 ml chloroform and washed with 60 ml 5% NaHCO3. The organic layer was separated and extracted with water (2×50 ml). The resulting organic layer was then dried and the DMT-O-methylpropan-3-ol was isolated by reverse phase chromatography. Compound I is converted to the phosphoramidite as described in example 1.a1.

### Example 6

### Alterations in Backbone

The methods of each of Examples 1 to 5 are repeated except that the oxidizer of Example 7 step 4 is substituted by a sulfur oxidizing agent such as Beaucage reagent (Glen Research Catalog No. 40-4036-XX) (Sterling, Va.) with the result that a phosphorothioate linkage is produced.

### Example 7

### Synthesis of Polymers Incorporating the Monomers of this Invention

The monomers of this application are incorporated into polymers via chemical processes commonly used for automated oligonucleotide synthesis. This method of synthesis utilizes a solid support that may or may not be covalently linked to the initial synthetic monomer prior to an initial deblocking step. The synthetic cycle consists of four steps: deblocking (detritylation), coupling, capping (A and B phases), and oxidation. Synthesis conditions for the polymer are performed at conditions compatible for the ABI Expedite DNA synthesizers (Life Technologies). All cycle and program times are as suggested by the manufacturer. Since DNA synthesizers do not generally have enough reagent slots for all of the monomers of this invention and other modifiers required for synthesis of chimeric polymers, partial sequences may be entered into different synthesizers with the final deblock step disabled. As each section of the polymer is completed, the column (containing the extended polymer still bound to solid support) is moved to the synthesizer programmed for the next section of the synthesis.

After each of the steps listed below, the growing support bound polymer is washed with anhydrous acetonitrile to remove unreacted chemicals and chemical byproducts.

Step 1: Deblocking

Deblocking removes the DMT group from the elongating terminus of the polymer under acidic conditions. A 3% dichloroacetic acid (DCA) solution, in an inert solvent DCM (dichloromethane) is used for deblocking. Deblock efficiency is monitored through generation of an orange reaction product. The result of deblocking is a free hydroxyl group at the point of polymer extension.

Step 2: Coupling

The dried nitrogen flushed monomer is suspended in anhydrous acetonitrile at a concentration of 0.02-0.2 M. The suspended monomer is attached to the synthesizer and flushed again briefly flushed with dry nitrogen or argon prior to synthesis (1 to 2 minutes). The monomer is activated by a 0.2-0.7 M 1H-tetrazole or 2-ethylthiotetrazole (or similar compatible compounds). Mixing of the monomer and activator is brief, occurring in fluid lines of the oligonucleotide synthesizer while the components are being delivered to the reaction column containing solid support. The activated phosphoramidite is supplied at a 1.5-20-fold excess over the support-bound material and reacts with the 5′-hydroxy group to form a phosphite triester linkage. Programmed reaction times for the monomers of this invention allow between 5 and 15 minutes for the coupling reaction. At the completion of each coupling step the reaction column is washed to remove unreacted material.

Step 3: Capping

To prevent extension of polymers where no monomer was added during the coupling step, the third step in the synthesis cycle is capping. Capping reagents include acetic anhydride and lmethyl amidizole or, in some cases, DMAP. These reagents react with the hydroxyl group to prevent further elongation. In cases where other reactive sites are present on the polymer the capping may prevent branching.

Step 4: Oxidation

Addition of the phosphoramidite monomer during the coupling step results in the formation of a phosphite triester linkage. When the growing polymer is treated with iodine in a weak base, such as pyridine or lutidine, (oxidizer) the linkage becomes a tetracoordinated phosphate triester that becomes a phosphodiester linkage on final deprotection of the polymer.

In cases where the final inter-monomer bonds are phosphorothioate, the sulfurization step is more efficiently performed prior to the capping step as step three, and the capping step then becomes step 4.

Final deblock, cleavage from the solid support, and deprotection

At the conclusion of synthesis, the polymer is deblocked a final time to remove the terminal DMT group. The polymer is then cleaved from the solid support under basic conditions, most commonly ammonia. The ammonia treatment also removes protecting groups on the polymer side chains (amino acid side chains or nucleotide bases).

### Example 7.1

### Incorporation of monomer subunit into a DNA Molecule

Following the method of Example 7, monomer subunits that are prepared in accordance with each of Examples 1 to 6 are incorporated into a DNA molecule having the following sequence AGTTGCACGT to obtain a synthetic polymeric molecule having the formula AGTTGCACGTM, wherein M represents the monomer subunit.

### Example 7.2

### Incorporation of Monomer Subunit into a DNA Molecule

Following the method of Example 7, monomer subunits that are prepared in accordance with each of Examples 1 to 6 are incorporated into a DNA molecule having the following sequence AGTTGCACGT to obtain a synthetic polymeric molecule having the formula AGTTGCACGTM, wherein M represents the monomer subunit, and then additional nucleotides are sequentially added to obtain a synthetic molecule having the formula AGTTGCACGTMCGAT.

### Example 7.3

### Incorporation of Monomer Subunit into an RNA Molecule

Following the method of Example 7, monomer subunits that are prepared in accordance with each of Examples 1 to 6 are incorporated into an RNA molecule having the following sequence AGUUGCACGU to obtain a synthetic polymeric molecule having the formula AGUUGCACGUM, wherein M represents the monomer subunit.

### Example 7.4

### Incorporation of Monomer Subunit into an RNA Molecule

Following the method of Example 7, monomer subunits that are prepared in accordance with each of Examples 1 to 6 are incorporated into an RNA molecule having the following sequence AGUUGCACGU to obtain a synthetic polymeric molecule having the formula AGUUGCACGUM, wherein M represents the monomer subunit, and then additional nucleotides are sequentially added to obtain a synthetic molecule having the formula AGUUGCACGUMCGAU.

### Example 7.5

### Homopolymer

Following the method of Example 7, a synthetic polymeric molecule containing ten consecutive monomers of Examples 1 to 5, each having the sidechain of histidine is prepared.

### Example 7.6

### Heteropolymer

Following the method of Example 7, a synthetic polymeric molecule containing thirty monomers of Examples 1 to 5, in which the amino acid sidechain of the monomers is varied is prepared.

### Example 7.7

### Heteropolymer Having a Non-Uniform Backbone

Following the method of Example 7, a synthetic polymeric molecule containing thirty monomers of Examples 1 to 5, in which the amino acid sidechain of the monomers is varied and in which the backbone group is varied is prepared.

### Example 7.8

### Heteropolymer Having a Non-Uniform Backbone and Non-Uniform Linker Group

Following the method of Example 7, a synthetic polymeric molecule containing twenty monomers of Examples 1 to 5, in which the amino acid sidechain of the monomers, the backbone group, and the linker (L) are varied is prepared.

### Example 8

### Use of Monomer

Example using monomer to produce a H-tag. Six His side chain monomers (H) as described in example 1 c2 are added during oligonucleotide synthesis to the 5′ position of a 20mer polydT, such that the final sequence is 5′HHH HHH TTT TTT TTT TTT TTT TTT TT3′. H is used at a concentration of 0.1M in acetonitrile for synthesis. The chimeric polymer is synthesized according to manufacturer's recommendations for the ABI Expedite synthesizer. After deprotection the H-tagged polydT was incubated with Histidine specific antibody (polyclonal IgG ab6442 from ABCAM). A control sample of 20-mer polydT was also incubated with the Histidine specific antibody. A native 12% polyacrylamide (20:1 acrylamide:bis) was loaded with 4 samples: 20-mer polydT, 20-mer polydT+Histidine specific antibody, His-tagged 20-mer polydT, His-tagged 20-mer polydT+Histidine specific antibody. (FICOLL® (GE Healthcare) added to all samples prior to loading onto the gel.) Addition of the Histidine specific antibody resulted in a gel shift for the His-tagged 20-mer polydT oligonucleotide as compared to the controls.

### Example 9

### Use of Monomer

The Cysteine side chain can form a disulfide bond capable of covalently linking two individual oligonucleotide single strands, once the monomer is incorporated into an oligonucleotide and deprotected. The sequence 5′TTT TTT TTT-C-TTT TTT TTT3′ where C represents a monomer of this invention with the cysteine side change used at a concentration of 0.1 M for synthesis. The chimeric molecule is synthesized according to manufacturer's recommendations for the ABI Expedite synthesizer.

A 50 nM scale synthesis of the chimeric polymer of this example is suspended in a volume of 20 microliters sterile water. One microliter of 10 mM DTT is added to 10 microliters of suspended polymer, and then held at room temperature for 10 minutes. The DTT is removed by short centrifugation in a Sephadex G-10 column, or by use of a push column. The resulting solution is evaporated to dryness under nitrogen. The dried sample is resuspended in 10 microliters water. A native 12% polyacrylamide (20:1 acrylamide:bis) was loaded with the following lanes: untreated chimeric polymer, DTT treated polymer, and 20-mer polydT oligonucleotide. (Ficoll added to all samples prior to loading onto the gel.) The resulting bands show a shift to higher apparent molecular mass in the gel for the chimeric sample allowed to dimerize.

### Example 10

### Uses of Synthetic Polymeric Molecule

Mimicking the Action of Arginine Vasopressin

Arginine vasopressin synthetic polymers according to the present application are made with the sequence of amino acid side chains of CYS-TYR-PHE-GLN-ASN-CYS-PRO-ARG-GLY. The sequence is replicated using the monomers of this application and standard DNA synthetic conditions for an ABI Expedite DNA Synthesizer.

Single VMCs from the left anterior descending, circumflex, and right coronary arteries of adult rhesus monkeys are isolated and studied both as freshly dispersed and as primary cultures. The short-term primary cultured cells (never passaged) maintain the characteristics of the source tissue for 2 to 3 weeks, including contraction, relaxation, receptors, and membrane electrical properties. VMCs are dissociated with collagenase and protease enzymes in a potassium glutamate solution (KG) that prevents loading with Na⁺, Ca2⁺, or Cl⁻ and results in a high proportion of viable, contracting cells (Miyagawa et al, Am J Physiol 272:H2645-2654, 1997; Hermsmeyer et, Art Thromb Vasc Biol, 24:955-961, 2004). The cells prepared for culture are seeded at low density in cardiovascular culture solution for mammals, fifth generation (CV5M) on glass coverslips to facilitate selection of individual cells. VMC are used for experiments 7-14 days after attaching to coverslips.

Freshly dispersed or primary cultured VMCs on glass coverslips are placed in a chamber of laminar flow design on an Axiovert inverted fluorescence microscope and observed with a Zeiss Plan Neofluar 25X/0.80 water immersion objective. Fluorescence intensity is measured with high sensitivity calibrated video sensor and recorded digitally via computer acquisition and management program. Ionic solution for mammals version 2 (ISM2) is continuously pumped through the chamber (at 1 ml/minute) to provide continuous equilibration and washout of agents. After a 15 minutes equilibration period, VMC are loaded for 15 minutes at room temperature with 3 μM fluo 3 (Molecular Probes, Inc.) for sensing Ca2⁺. Individual VMC are stimulated by adding arginine vasopressin or arginine vasopressin analog of this application over the individual cell. After 15 seconds under no flow conditions, continuous flow of ISM2 is reinstated and a chamber volume of approximately 300 μl is maintained. Fluorescent images are taken at 1 minute and subsequent time points from which light intensity is equated to changes in calcium signal (fluorescence) and cell contractility. It is found that polymers of the invention affect calcium signal and cell contractility in the VMC as does the native arginine vasopressin.

Various modifications of the above described invention will be evident to those skilled in the art. It is intended that such modifications are included within the scope of the following claims.

1. A synthetic polymeric molecule comprising a multiplicity of monomer subunits wherein at least one of the monomer subunits contains an amino acid side chain linked to a phosphate or modified phosphate group, and wherein the phosphate or modified phosphate group of the monomer subunit is linked to an adjacent monomer subunit by a phosphodiester or modified phosphodiester bond.

2. The synthetic polymeric molecule of claim 1 wherein the monomer subunit containing an amino acid side chain linked to a phosphate or modified phosphate group contains a sugar moiety between the amino acid side chain and the phosphate or modified phosphate group.

3. The synthetic polymeric molecule of claim 2 wherein the sugar moiety is a pentose sugar moiety.

4. The synthetic polymeric molecule of claim 3 wherein the pentose sugar moiety is a deoxyribose or ribose sugar moiety.

5. The synthetic polymeric molecule of claim 1 wherein the monomer subunits that contains an amino acid side chain linked to a phosphate or modified phosphate group is linked to the adjacent monomer by a phosphodiester bond.

6. The synthetic polymeric molecule of claim 1 wherein the monomer that contains an amino acid side chain linked to a phosphate or modified phosphate group is linked to the adjacent monomer by a modified phosphodiester bond.

7. The synthetic polymeric molecule of claim 6 wherein the modified phosphodiester bond is selected from the group consisting of phosphorothiolate, diphosphorothiolate, alkylphosphonate, alkoxyphosphonate, and phosphoramidate bonds.

8. The synthetic polymeric molecule of claim 1 wherein the monomer subunit that contains an amino acid side chain linked to a phosphate or modified phosphate group has the formula: 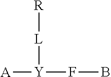
wherein:
A=H, OH, an alkyl group, an alkoxy group, an amino group, a carboxy group, biotin, a dye, a reversed linkage, an amino acid, a polypeptide or analog, a nucleotide, an oligonucleotide, a solid support, a linkage to a solid support, or an adjacent monomer subunit,
B=H, OH, an alkyl group, an alkoxy group, an amino group, a carboxy group, biotin, a dye, a reversed linkage, an amino acid, a polypeptide or analog, a nucleotide, an oligonucleotide, a solid support, a linkage to a solid support, or an adjacent monomer subunit,
L=a linker that is covalently bound to R,
Y=a spacer between L and F,
F=a phosphodiester or modified phosphodiester group, and
R=an amino acid side chain.

9. The synthetic polymeric molecule of claim 1 wherein each of the monomeric subunits of the molecule contains an amino acid side chain linked to a phosphate or modified phosphate group, and wherein the phosphate or modified phosphate group of the monomer subunit is linked to an adjacent monomer subunit by a phosphodiester or modified phosphodiester bond.

10. The synthetic polymeric molecule of claim 1 wherein one or more of the monomeric subunits of the molecule is other than a subunit that contains an amino acid side chain linked to a phosphate or modified phosphate group, and wherein the phosphate or modified phosphate group of the monomer subunit is linked to an adjacent monomer subunit by a phosphodiester or modified phosphodiester bond.

11. The synthetic polymeric molecule of claim 1 wherein one or more of the monomeric subunits are protected by the presence of one or more protecting groups.

12. A monomer molecule comprising an amino acid side chain other than that of glycine linked to a phosphate or modified phosphate group.

13. The monomer molecule of claim 12 that comprises a sugar moiety between the amino acid side chain and the phosphate or modified phosphate group.

14. The monomer molecule of claim 13 wherein the sugar moiety is a pentose sugar moiety.

15. The monomer molecule of claim 14 wherein the pentose sugar moiety is a deoxyribose or ribose sugar moiety.

16. (canceled)

17. The monomer molecule of claim 12 wherein the amino acid side chain is linked to a modified phosphate group.

18. The monomer molecule of claim 17 wherein the modified phosphate group is selected from the group consisting of phosphoramidate, phosphoramidite, phosphonate, phosphorothioate, phosphorodithioate, phosphorothiolate, diphosphorothiolate, alkylphosphonate, alkoxyphosphonate, and alkoxy groups.

19. The monomer molecule of claim 12 which is protected by the presence of one or more protecting groups.

20. The monomer molecule of claim 12 that has the formula: 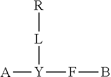
wherein:
A=H, OH, an alkyl group, an alkoxy group, an amino group, a carboxy group, biotin, a dye, a reversed linkage, an amino acid, a polypeptide or analog, a nucleotide, an oligonucleotide, a solid support, or a linkage to a solid support,
B=H, OH, an alkyl group, an alkoxy group, an amino group, a carboxy group, biotin, a dye, a reversed linkage, an amino acid, a polypeptide or analog, a nucleotide, an oligonucleotide, a solid support, or a linkage to a solid support,
L=a linker that is covalently bound to R,
Y=a spacer between L and F,
F=a phosphodiester or modified phosphodiester group, and
R=an amino acid side chain.

21. A method for elongating a synthetic polymeric molecule comprising obtaining a monomer molecule of claim 12 and linking the monomer molecule to a monomer subunit of the synthetic polymeric molecule by a phosphodiester or modified phosphodiester bond.


SEQUENCE LISTING (9 sequences)

SEQ ID NO: 1 - DNA, 10 bp, Artificial Sequence
  Synthesized
agttgcacgt

SEQ ID NO: 2 - DNA, 11 bp, Artificial Sequence
  Synthesized
agttgcacgtm

SEQ ID NO: 3 - DNA, 15 bp, Artificial Sequence
  Synthesized
agttgcacgtmcgat

SEQ ID NO: 4 - RNA, 10 nt, Artificial Sequence
  Synthesized
aguugcacgu

SEQ ID NO: 5 - RNA, 11 nt, Artificial Sequence
  Synthesized
aguugcacgum

SEQ ID NO: 6 - RNA, 15 nt, Artificial Sequence
  Synthesized
aguugcacgumcgau

SEQ ID NO: 7 - DNA, 26 bp, Artificial Sequence
  Synthesized
hhhhhhtttttttttttttttttttt

SEQ ID NO: 8 - DNA, 19 bp, Artificial Sequence
  Synthesized
tttttttttcttttttttt

SEQ ID NO: 9 - protein, 9 aa, Homo sapiens
CYFQNCPRG